(19) United States
(12) Patent Application Publication
(10) Pub. No.: US 2026/0184747 A1
(43) Pub. Date: Jul. 2, 2026

(54) SWEET PROTEIN MUTANTS, PREPARATION METHODS, AND USE THEREOF

(71) Applicants: KINGDOMWAY BIOTECHNOLOGY (JIANGSU) CO., LTD., Nantong (CN); INNER MONGOLIA KINGDOMWAY PHARMACEUTICAL CO., LTD., Huhhot (CN); XIAMEN KINGDOMWAY GROUP COMPANY, Xiamen (CN)

(72) Inventors: Jun ZHU, Nantong (CN); Fei XU, Nantong (CN); Xingyi ZHANG, Nantong (CN); Deyao MENG, Nantong (CN); Dan LI, Xiamen (CN); Faguang LIU, Nantong (CN)

(73) Assignees: KINGDOMWAY BIOTECHNOLOGY (JIANGSU) CO., LTD., Nantong (CN); INNER MONGOLIA KINGDOMWAY PHARMACEUTICAL CO., LTD., Huhhot (CN); XIAMEN KINGDOMWAY GROUP COMPANY, Xiamen (CN)

(21) Appl. No.: 19/214,031

(22) Filed: May 20, 2025

(30) Foreign Application Priority Data
Dec. 31, 2024 (CN) 202411987663.7

(51) Int. Cl. C07K 14/43; C12N 15/81; C12P 21/02

(52) U.S. Cl. CPC C07K 14/43; C12N 15/815; C12P 21/02; C12N 2800/102

(57) ABSTRACT

Sweet protein mutants, preparation methods, and use thereof are provided. The sweet protein mutant is obtained by inducing one or more amino acid mutations in a sweet protein with an amino acid sequence shown in SEQ ID NO. 1, the one or more amino acid mutations including at least one of K5D, V7R, D29N, or E53R. By introducing mutations into the amino acid sequence of the sweet protein, the sweetness of the sweet protein mutant is significantly improved compared with the sweet protein corresponding to the original amino acid sequence.

### CROSS-REFERENCE TO RELATED APPLICATIONS

This application claims priority to Chinese Patent Application No. 202411987663.7, filed on Dec. 31, 2024, the entire contents of which are incorporated herein by reference.

### SEQUENCE LISTING

The instant application contains a Sequence Listing which is submitted electronically in XML format and is hereby incorporated by reference in its entirety. The XML copy, created on Mar. 27, 2025, is named “2025 Mar. 27-Sequence List-20605-0005US00,” and is 23,704 bytes in size.

### TECHNICAL FIELD

The present disclosure relates to the field of biotechnology, and in particular to a sweet protein mutant and a preparation method and use thereof.

### BACKGROUND

The World Health Organization (WHO) indicates that excessive sugar intake is a significant inducement for chronic diseases such as obesity and diabetes. According to the standards announced by the WHO in 2019, the daily sugar intake for adults should not exceed 25 g. With the changes in people's living habits, the sugar substitute industry has gradually emerged, leading to the rise of suppliers specializing in “sugar-free products”.

As a substitute for sugar, sweeteners have garnered much attention. In recent years, some companies have begun to focus on sweet proteins, a type of high-intensity, pure-tasting, and natural sweeteners free of side effects, which are expected to lead the future of the high-intensity sweetener industry. Due to its sugar-like taste, absence of off-flavors, and structural stability in a wide range of temperatures and pH, brazzein protein is considered as the most promising sweet protein among various sweet proteins. Brazzein protein is extracted from the fruit of the West African tropical plant Pentadiplandra brazzeana Baillon and it exhibits a sweetness intensity 500-2000 times that of the equal mass of sucrose. Compared with other sweet proteins, the brazzein protein has advantages of the smallest molecular weight, excellent water solubility, and retaining its sweetness after heat treatment at 80° C. for 4 h, demonstrating good thermal stability and pH stability.

However, the yield of the sweet protein derived from natural plants is relatively low, making it difficult to meet market demand. In addition, the sweet protein faces issues such as misfolding, tendency to form inclusion bodies, and insufficient expression in heterologous recombinant expression, which hinders its commercial production and application.

### SUMMARY

One or more embodiments of the present disclosure provide a sweet protein mutant. The sweet protein mutant is obtained by inducing one or more amino acid mutations in a sweet protein with an amino acid sequence shown in SEQ ID NO. 1, the one or more amino acid mutations including at least one of K5D, V7R, D29N, or E53R.

One or more embodiments of the present disclosure provide a nucleic acid molecule encoding the sweet protein mutant.

One or more embodiments of the present disclosure provide an expression vector comprising at least one copy of the nucleic acid molecule.

One or more embodiments of the present disclosure provide a sweet protein mutant transformant. The sweet protein mutant transformant is a genetically engineered strain that expresses the sweet protein mutant.

One or more embodiments of the present disclosure provide a preparation method for the sweet protein mutant. The preparation method comprises constructing an expression vector, the expression vector comprising at least one copy of a nucleic acid sequence that encodes the sweet protein mutant; constructing a sweet protein mutant transformant by transforming the expression vector into a recipient cell; and obtaining the sweet protein mutant by culturing the sweet protein mutant transformant and collecting a culture.

One or more embodiments of the present disclosure provide a purification method for the sweet protein mutant. The purification method comprises: culturing a sweet protein mutant transformant to collect a culture, separating and removing contaminating proteins from the culture and filtering, adsorbing the sweet protein mutant from a filtered solution through a cation exchange resin, followed by eluting to collect an eluent, performing nanofiltration on the eluent for desalination and concentration, and performing lyophilization on a nanofiltered eluent to obtain a purified sweet protein mutant, the sweet protein mutant transformant comprising a nucleic acid molecule encoding the sweet protein mutant.

### DETAILED DESCRIPTION

In order to more clearly illustrate the technical solutions of the embodiments of the present disclosure, a brief introduction to the embodiments is given below.

As shown in the present disclosure and in the claims, unless the context clearly suggests an exception, the words “one,” “a,” “an,” “one kind,” and/or “the” do not refer specifically to the singular, but may also include the plural. Generally, the terms “including” and “comprising” suggest only the inclusion of clearly identified steps and elements, however, the steps and elements that do not constitute an exclusive list, and the method or apparatus may also include other steps or elements.

In view of the deficiencies in the prior art, the present disclosure provides sweet protein mutants, preparation methods, and use thereof. According to the present disclosure, the sweet protein gene is artificially and targeted modified using genetic engineering techniques, which significantly improves the sweetness of sweet protein.

The embodiments of the present disclosure provide a sweet protein mutant. The sweet protein mutant is obtained by inducing one or more amino acid mutations in a sweet protein with an amino acid sequence shown in SEQ ID NO. 1, the one or more amino acid mutations including at least one of K5D, V7R, D29N, or E53R. The K5D amino acid mutation is a mutation of lysine (K) at the 5th position of the amino acid sequence to aspartic acid (D), the V7R amino acid mutation is a mutation of valine (V) at the 7th position of the amino acid sequence to arginine (R), the D29N amino acid mutation is a mutation of aspartic acid (D) at the 29th position of the amino acid sequence to asparagine (N), and the E53R amino acid mutation is a mutation of glutamic acid (E) at the 53rd position of the amino acid sequence to arginine (R).

In some embodiments of the present disclosure, the sweet protein mutant is obtained by introducing the one or more amino acid mutations in the sweet protein with the amino acid sequence shown in SEQ ID NO. 1. Compared with sweet protein corresponding to an original amino acid sequence, the relative sweetness of the sweet protein mutant is significantly improved.

In some embodiments, the one or more amino acid mutations are at least one of K5D, V7R, D29N, E53R, K5D+V7R, D29N+E53R, or K5D+V7R+D29N+E53R. In some embodiments, the one or more amino acid mutations are at least one of E53R, D29N+E53R, or K5D+V7R+D29N+E53R.

SEQ ID NO. 1:
QDKCKKVYENYPVSKCQLANQCNYDCKLDKHARSGEC
FYDEKRNLQCICDYCEY.

In some embodiments, an unmutated sweet protein (i.e., an original sweet protein) is encoded by a nucleic acid sequence as set forth in SEQ ID NO. 2.

SEQ ID NO. 2:
CAGGACAAGTGCAAGAAGGTCTACGAGAACTACCCAGTTTCCAAGT
GCCAGTTGGCTAACCAGTGTAACTACGACTGCAAGTTGGACAAGC
ACGCTAGATCTGGTGAGTGTTTCTACGACGAGAAGAGAAACCTGC
AGTGCATCTGTGACTACTGCGAGTACTAA.

In some embodiments, an amino acid sequence of a K5D mutant is shown in SEQ ID NO. 11, and a nucleic acid sequence encoding the K5D mutant is shown in SEQ ID NO. 12.

SEQ ID NO. 11:
QDKCDKVYENYPVSKCQLANQCNYDCKLDKHARSGECFYDEKRNLQCICD
YCEY.
SEQ ID NO. 12:
CAGGACAAGTGCGACAAGGTCTACGAGAACTACCCAGTTTCCAAGTGCCA
GTTGGCTAACCAGTGTAACTACGACTGCAAGTTGGACAAGCACGCTAGAT
CTGGTGAGTGTTTCTACGACGAGAAGAGAAACCTGCAGTGCATCTGTGAC
TACTGCGAGTACTAA.

In some embodiments, an amino acid sequence of a V7R mutant is shown in SEQ ID NO. 13, and a nucleic acid sequence encoding the V7R mutant is shown in SEQ ID NO. 14.

SEQ ID NO. 13:
QDKCKKRYENYPVSKCQLANQCNYDCKLDKHARSGECFYDEKRNLQCICD
YCEY.
SEQ ID NO. 14:
CAGGACAAGTGCAAGAAGCGCTACGAGAACTACCCAGTTTCCAAGTGCCA
GTTGGCTAACCAGTGTAACTACGACTGCAAGTTGGACAAGCACGCTAGAT
CTGGTGAGTGTTTCTACGACGAGAAGAGAAACCTGCAGTGCATCTGTGAC
TACTGCGAGTACTAA.

In some embodiments, an amino acid sequence of a D29N mutant is shown in SEQ ID NO. 15, and a nucleic acid sequence encoding the D29N mutant is shown in SEQ ID NO. 16.

SEQ ID NO. 15:
QDKCKKVYENYPVSKCQLANQCNYDCKLNKHARSGECFYDEKRNLQCICD
YCEY.
SEQ ID NO. 16:
CAGGACAAGTGCAAGAAGGTCTACGAGAACTACCCAGTTTCCAAGTGCCA
GTTGGCTAACCAGTGTAACTACGACTGCAAGTTGAACAAGCACGCTAGAT
CTGGTGAGTGTTTCTACGACGAGAAGAGAAACCTGCAGTGCATCTGTGAC
TACTGCGAGTACTAA.

In some embodiments, an amino acid sequence of an E53R mutant is shown in SEQ ID NO. 17, and a nucleic acid sequence encoding the E53R mutant is shown in SEQ ID NO. 18.

SEQ ID NO. 17:
QDKCKKVYENYPVSKCQLANQCNYDCKLDKHARSGECFYDEKRNLQCICD
YCRY.
SEQ ID NO. 18:
CAGGACAAGTGCAAGAAGGTCTACGAGAACTACCCAGTTTCCAAGTGCCA
GTTGGCTAACCAGTGTAACTACGACTGCAAGTTGGACAAGCACGCTAGAT
CTGGTGAGTGTTTCTACGACGAGAAGAGAAACCTGCAGTGCATCTGTGAC
TACTGCAGATACTAA.

In some embodiments, an amino acid sequence of a K5D+V7R mutant is shown in SEQ ID NO. 19, and a nucleic acid sequence encoding the K5D+V7R mutant is shown in SEQ ID NO. 20.

SEQ ID NO. 19:
QDKCDKRYENYPVSKCQLANQCNYDCKLDKHARSGECFYDEKRNLQCICD
YCEY.
SEQ ID NO. 20:
CAGGACAAGTGCGACAAGCGCTACGAGAACTACCCAGTTTCCAAGTGCCA
GTTGGCTAACCAGTGTAACTACGACTGCAAGTTGGACAAGCACGCTAGAT
CTGGTGAGTGTTTCTACGACGAGAAGAGAAACCTGCAGTGCATCTGTGAC
TACTGCGAGTACTAA.

In some embodiments, an amino acid sequence of a D29N+E53R mutant is shown in SEQ ID NO. 21, and a nucleic acid sequence encoding the D29N+E53R mutant is shown in SEQ ID NO. 22.

SEQ ID NO. 21:
QDKCKKVYENYPVSKCQLANQCNYDCKLNKHARSGECFYDEKRNLQCICD
YCRY.
SEQ ID NO. 22:
CAGGACAAGTGCAAGAAGGTCTACGAGAACTACCCAGTTTCCAAGTGCCA
GTTGGCTAACCAGTGTAACTACGACTGCAAGTTGAACAAGCACGCTAGAT
CTGGTGAGTGTTTCTACGACGAGAAGAGAAACCTGCAGTGCATCTGTGAC
TACTGCAGATACTAA.

In some embodiments, an amino acid sequence of a K5D+V7R+D29N+E53R mutant is shown in SEQ ID NO. 23, and a nucleic acid sequence encoding the K5D+V7R+D29N+E53R mutant is shown in SEQ ID NO. 24.

SEQ ID NO. 23:
QDKCDKRYENYPVSKCQLANQCNYDCKLNKHARSGECFYDEKRNLQCICD
YCRY.
SEQ ID NO. 24:
CAGGACAAGTGCGACAAGCGCTACGAGAACTACCCAGTTTCCAAGTGCCA
GTTGGCTAACCAGTGTAACTACGACTGCAAGTTGAACAAGCACGCTAGAT
CTGGTGAGTGTTTCTACGACGAGAAGAGAAACCTGCAGTGCATCTGT.

In some embodiments, the unmutated sweet protein is derived from the West African tropical plant Pentadiplandra brazzeana Baillon.

The embodiments of the present disclosure provide a nucleic acid molecule encoding a sweet protein mutant.

The embodiments of the present disclosure provide an expression vector comprising at least one copy of a nucleic acid molecule.

The embodiments of the present disclosure provide a sweet protein mutant transformant. The sweet protein mutant transformant is a genetically engineered strain that expresses a sweet protein mutant.

The embodiments of the present disclosure provide a preparation method for a sweet protein mutant. The preparation method comprises constructing an expression vector, the expression vector comprising at least one copy of a nucleic acid sequence that encodes the sweet protein mutant; constructing a sweet protein mutant transformant by transforming the expression vector into a recipient cell; and obtaining the sweet protein mutant by culturing the sweet protein mutant transformant and collecting a culture.

In some embodiments, the preparation method for the sweet protein mutant further comprises: synthesizing a nucleic acid fragment encoding a sweet protein derived from a West African tropical plant Pentadiplandra brazzeana Baillon via whole gene synthesis, the nucleic acid fragment having a sequence as set forth in SEQ ID NO. 2, and ligating the nucleic acid fragment into an expression vector via restriction enzyme digestion to obtain a recombinant plasmid; performing site-directed mutagenesis using the recombinant plasmid as a template to obtain the expression vector comprising at least one copy of the nucleic acid sequence of the sweet protein mutant; and transforming the expression vector comprising at least one copy of the nucleic acid sequence of the sweet protein mutant into a host cell for culturing to obtain the sweet protein mutant.

In some embodiments, the host cell is Pichia pastoris X-33.

The embodiments of the present disclosure provide a purification method for a sweet protein mutant. The purification method comprises: culturing a sweet protein mutant transformant to collect a culture, separating and removing contaminating proteins from the culture and filtering, adsorbing the sweet protein mutant from a filtered solution through a cation exchange resin, followed by eluting to collect an eluent, performing nanofiltration on the eluent for desalination and concentration, and performing lyophilization on a nanofiltered eluent to obtain a purified sweet protein mutant, the sweet protein mutant transformant comprising a nucleic acid molecule encoding the sweet protein mutant.

In some embodiments, the contaminating proteins are separated and removed from the culture via centrifugal separation.

In some embodiments, the filtering is performed using a 0.5-10 kDa ultrafiltration membrane. In some embodiments, the filtering is performed using a 0.5 kDa ultrafiltration membrane, a 1 kDa ultrafiltration membrane, a 2 kDa ultrafiltration membrane, a 3 kDa ultrafiltration membrane, a 4 kDa ultrafiltration membrane, a 5 kDa ultrafiltration membrane, a 6 kDa ultrafiltration membrane, a 7 kDa ultrafiltration membrane, an 8 kDa ultrafiltration membrane, a 9 kDa ultrafiltration membrane, or a 10 kDa ultrafiltration membrane.

In some embodiments, the eluting is performed using a NaCl aqueous solution.

In some embodiments, in the NaCl aqueous solution, a NaCl concentration is within a range of 1-2 M. In some embodiments, the NaCl concentration in the NaCl aqueous solution is 1 M, 1.3 M, 1.5 M, 1.8 M, or 2 M. In some embodiments, a pH of the NaCl aqueous solution is within a range of 3.0-7.0. In some embodiments, the pH of the NaCl aqueous solution is 3.0, 3.5, 4.0, 4.5, 5.0, 5.5, 6.0, 6.5, or 7.0.

In some embodiments of the present disclosure, the sweet protein mutant is obtained by performing site-directed mutagenesis on the amino acid sequence of the sweet protein. Compared with the original sweet protein, the sweetness of the sweet protein mutant is significantly improved, and the sweetness of the sweet protein mutant is 1.6-6 times that of the original sweet protein.

The technical solution of the present disclosure will be further described in detail with reference to the specific examples. It should be understood that the following examples are only intended to exemplify and explain the present disclosure and should not be construed as limiting the scope of protection of the present disclosure. All technologies implemented based on the above contents of the present disclosure are included in the scope of protection intended by the present disclosure.

### EXAMPLES

### Example 1 Construction of Brazzein Recombinant Plasmid

Sangon Biotech (Shanghai) Co., Ltd. synthesized a nucleic acid fragment encoding a brazzein protein derived from a West African tropical plant Pentadiplandra brazzeana Baillon via whole gene synthesis, the nucleic acid fragment having a sequence as set forth in SEQ ID NO. 2, and ligated the nucleic acid fragment into an expression vector pPICZαA via a DNA ligase after digestion with restriction enzymes XhoI and NotI (purchased from New England Biolabs and operated according to the instructions) to obtain a recombinant plasmid Brazzein-pPICZαA.

### Example 2 Construction of Brazzein Mutant

The site-directed mutagenesis was performed using the recombinant plasmid Brazzein-pPICZαA as a template using forward/reverse mutation primers of K5D, V7R, D29N, and E53R, respectively via the Fast Mutagenesis System of the TransGen Biotech to obtain the brazzein mutant.

Where
lysine (K) at the 5th position of the amino acid sequence was mutated to aspartic acid (D),
a nucleic acid sequence of the forward mutation primer K5D-F was as set forth in

SEQ ID NO. 3:
5′ GAAAAGACAGGACAAGTGCGACAAGGTCTACGAGAACTAC 3′;

a nucleic acid sequence of the reverse mutation primer K5D-R was as set forth in SEQ ID NO. 4:

5′ GTAGTTCTCGTAGACCTTGTCGCACTTGTCCTGTCTTTTC 3′;

valine (V) at the 7th position of the amino acid sequence was mutated to arginine (R),
a nucleic acid sequence of the forward mutation primer V7R-F was as set forth in

SEQ ID NO. 5:
5′ GACAAGTGCAAGAAGCGCTACGAGAACTACC 3′;

a nucleic acid sequence of the reverse mutation primer V7R-R was as set forth in SEQ ID NO. 6:

5′ GGTAGTTCTCGTAGCGCTTCTTGCACTTGTC3′;

aspartic acid (D) at the 29th position of the amino acid sequence was mutated to asparagine (N),
a nucleic acid sequence of the forward mutation primer D29N-F was as set forth in SEQ ID NO. 7:

5′ CTACGACTGCAAGTTGAACAAGCACGCTAGATC 3′;

a nucleic acid sequence of the reverse mutation primer D29N-R was as set forth in SEQ ID NO. 8:

5′ GATCTAGCGTGCTTGTTCAACTTGCAGTCGTAG 3′;

glutamic acid (E) at the 53rd position of the amino acid sequence was mutated to arginine (R),
a nucleic acid sequence of the forward mutation primer E53R-F was as set forth in SEQ ID NO. 9:

5′ ATCTGTGACTACTGCAGATACTAAGCG 3′;

a nucleic acid sequence of the reverse mutation primer E53R-R was as set forth in SEQ ID NO. 10:

5′ TCTGCAGTAGTCACAGATGCACTGCAG 3′.

A PCR system contained 25 μL of 2× TransStart® FastPfu Fly PCR SuperMix, 1 μL of 10 UM forward primer and 1 μL of 10 UM reverse primer, and 1 μL of 50 ng/μL template plasmid, ddH₂O was supplemented to 50 μL. A PCR amplification process comprised: performing pre-denaturation at a temperature of 94° C. for 3 min followed by 30 cycles of: denaturation at the temperature of 94° C. for 20 s, annealing at the temperature of 55° C. for 20 s, extending at the temperature of 72° C. for 2 min; and finally extending at the temperature of 72° C. for 10 min. A PCR amplification product was digested with DpnI for 2 h, a digested product was transformed into E. coli Tran5α and preserved after monoclonal sequencing for verification.

### Example 3 Expression of Brazzein Recombinant Plasmid and Brazzein Mutant in Pichia pastoris

The recombinant plasmid Brazzein-pPICZαA in Example 1 and the brazzein mutant in Example 2 were linearized with SacI, respectively. The linearized products were purified by a column purification kit, transformed into Pichia pastoris X-33 by electroporation, and spread on YPD+Zeocin (100 mg/L) plates. Strains grown on the YPD+Zeocin (100 mg/L) plates were engineered Pichia pastoris strains. Verification by shake-flask fermentation comprised: selecting transformants to be inoculated into a buffered glycerol-complex (BMGY) medium for shaking cultivation at 30° C. and 220 rpm for 24 h, then transferring to a buffered methanol-complex (BMMY) medium for shaking cultivation at 30° C. and 220 rpm, adding 0.5% methanol every 24 h to induce expression, and after 4 d, performing centrifugal separation to remove the strains and collecting a fermentation supernatant containing the brazzein protein.

### Example 4 Purification of Brazzein Protein and Brazzein Mutant

### (a) Pretreatment of the Fermentation Supernatant

8000 g of the fermentation supernatant containing the brazzein protein obtained in Example 3 was centrifugally separated for 30 min to collect a supernatant solution.

### (b) Crude Treatment of the Fermentation Supernatant

The supernatant solution after the pretreatment in step (a) was ultrafiltered using a 1 kDa ultrafiltration membrane for concentration and desalination. During the concentration, pure water was continuously added to dilute a salt concentration in a concentrated solution until its conductivity is less than 0.1 mS/m.

### (c) Purification of the Brazzein Protein

Protein purification resin column: specification: 10×30 cm (1L); filler: CM cation resin (6% cross-linked agarose, purchased from Suzhou Zhongke Senhui Microsphere Technology).

Equilibration: the resin column was equilibrated with 3 L acetic acid aqueous solution with a pH of 4.0 at a flow rate of 10 mL/min.

Sample loading: 5 L sample after the crude treatment in step (b) was loaded at a flow rate of 5 mL/min.

Washing: the sample was washed with the 3 L acetic acid aqueous solution with a pH of 4.0 at the flow rate of 10 mL/min.

Elution: the sample was eluted with 3 L 1 M acetic acid solution of NaCl at the flow rate of 10 mL/min to obtain an eluate containing the brazzein protein.

### (d) Concentration and Desalination of the Eluate

The eluate obtained in step (c) was filtered through a nanofiltration membrane (1000 Da, Shanghai Langji Membrane Separation Equipment Engineering Co., Ltd.) for desalination to obtain a concentrated sample.

### (e) Lyophilization of the Concentrated Sample

Excess water of the concentrated sample obtained in step (d) was removed using a lyophilizer to obtain a dried brazzein protein sample.

### Example 5 Sensory Evaluation of Brazzein Protein and Brazzein Mutant

The sweetness of the sweet protein was verified using a blind test. A control group of the blind test used a 2% sucrose aqueous solution. The sweet protein samples to be tested were prepared into different concentration gradients of solutions and these solutions were randomly numbered. An evaluation team consisting of ten persons tasted the solutions and determined a solution with the same or similar sweetness as the 2% sucrose aqueous solution in the different concentration gradients of solutions, so as to calculate the relative sweetness of the samples.

Firstly, 1.0 g sweet protein sample was weighed and diluted to 100 mL with distilled water, i.e., a sample solution with a concentration of 1.0% was prepared; then 1.0% sample solution was taken and further diluted with the distilled water to form test solutions with different dilution multiples, whose concentration was 0.5%, 0.25%, 0.2%, 0.125%, 0.1%, 0.05%, 0.025%, and 0.0125%, respectively. The sweet protein samples to be tested were prepared into different concentration gradients of solutions and these solutions were randomly numbered. An evaluation team consisting of ten persons tasted the solutions and determined a solution with the same or similar sweetness as the 2% sucrose aqueous solution in the different concentration gradients of solutions, so as to calculate the relative sweetness of the samples. The formula is expressed as: the relative sweetness=2% sucrose aqueous solution/(1.0% sample solution×dilution multiple).

The test results are shown in Table 1.

TABLE 1

| | Relative sweetness |
|---|---|
| | (multiple relative to |
| | the sweetness of 2% |
| Sweet protein | sucrose aqueous solution) |
| WT | 500 |
| K5D | 800 |
| V7R | 1200 |
| D29N | 1500 |
| E53R | 1000 |
| K5D + V7R | 1500 |
| D29N + E53R | 2000 |
| K5D + V7R + | 3000 |
| D29N + E53R | |

As can be seen from the above that the sweetness of the brazzein mutant of the present disclosure is 1.6-6 times that of the original brazzein protein, and its sweetness is significantly improved.

The present disclosure illustrates the sweet protein mutants, the preparation methods, and the use thereof with reference to the embodiments, but the present disclosure is not limited to the embodiments. That is, it does not mean that the present disclosure must rely on the embodiments to be implemented. Those skilled in the art should understand that any improvement to the present disclosure, equivalent replacement of various raw materials of the products of the present disclosure, addition of auxiliary ingredients, selection of specific implementations, etc., fall within the protection scope and disclosure scope of the present disclosure.

1. A sweet protein mutant, wherein the sweet protein mutant is obtained by inducing one amino acid mutations in a sweet protein with an amino acid sequence shown in SEQ ID NO. 1, the amino acid mutations are K5D+V7R+D29N+E53R.

2. The sweet protein mutant of claim 1, wherein the sweet protein is encoded by a nucleic acid sequence as set forth in SEQ ID NO. 2.

3-4. (canceled)

5. The sweet protein mutant of claim 1, wherein
an amino acid sequence of a K5D+V7R+D29N+E53R mutant is shown in SEQ ID NO. 23, and a nucleic acid sequence encoding the K5D+V7R+D29N+E53R mutant is shown in SEQ ID NO. 24.

6. A nucleic acid molecule encoding the sweet protein mutant of claim 1.

7. An expression vector comprising at least one copy of the nucleic acid molecule of claim 6.

8. A sweet protein mutant transformant, wherein the sweet protein mutant transformant is a genetically engineered strain that expresses the sweet protein mutant of claim 1.

9. A preparation method for the sweet protein mutant of claim 1, comprising:
constructing an expression vector, wherein the expression vector comprises at least one copy of a nucleic acid sequence that encodes the sweet protein mutant;
constructing a sweet protein mutant transformant by transforming the expression vector into a recipient cell; and
obtaining the sweet protein mutant by culturing the sweet protein mutant transformant and collecting a culture.

10. The preparation method of claim 9, further comprising:
synthesizing a nucleic acid fragment encoding a sweet protein derived from a West African tropical plant Pentadiplandra brazzeana Baillon via whole gene synthesis, the nucleic acid fragment having a sequence as set forth in SEQ ID NO. 2, and ligating the nucleic acid fragment into an expression vector via restriction enzyme digestion to obtain a recombinant plasmid;
performing site-directed mutagenesis using the recombinant plasmid as a template to obtain the expression vector comprising at least one copy of the nucleic acid sequence of the sweet protein mutant; and
transforming the expression vector comprising at least one copy of the nucleic acid sequence of the sweet protein mutant into a host cell for culturing to obtain the sweet protein mutant.

11. A purification method for the sweet protein mutant of claim 1, comprising:
culturing a sweet protein mutant transformant to collect a culture, separating and removing contaminating proteins from the culture and filtering, adsorbing the sweet protein mutant from a filtered solution through a cation exchange resin, followed by eluting to collect an eluent, performing nanofiltration on the eluent for desalination and concentration, and performing lyophilization on a nanofiltered eluent to obtain a purified sweet protein mutant, wherein the sweet protein mutant transformant comprises a nucleic acid molecule encoding the sweet protein mutant.

12. The purification method of claim 11, wherein the contaminating proteins are separated and removed from the culture via centrifugal separation.

13. The purification method of claim 11, wherein the filtering is performed using a 0.5-10 kDa ultrafiltration membrane.

14. The purification method of claim 11, wherein the eluting is performed using a NaCl aqueous solution.

15. The purification method of claim 14, wherein in the NaCl aqueous solution, a NaCl concentration is within a range of 1-2 M, and a pH is within a range of 3.0-7.0.


SEQUENCE LISTING (24 sequences)

SEQ ID NO: 1 - protein, 54 aa, unidentified
QDKCKKVYENYPVSKCQLANQCNYDCKLDKHARSGECFYDEKRNLQCICDYCEY

SEQ ID NO: 2 - DNA, 165 bp, unidentified
  source 1..165: mol_type = other DNA
caggacaagtgcaagaaggtctacgagaactacccagtttccaagtgccagttggctaaccagtgtaactacgactgcaagttggacaagcacgctagatctggtgagtgtttctacgacgagaagagaaacctgcagtgcatctgtgactactgcgagtactaa

SEQ ID NO: 3 - DNA, 40 bp, synthetic construct
  source 1..40: mol_type = other DNA
gaaaagacaggacaagtgcgacaaggtctacgagaactac

SEQ ID NO: 4 - DNA, 40 bp, synthetic construct
  source 1..40: mol_type = other DNA
gtagttctcgtagaccttgtcgcacttgtcctgtcttttc

SEQ ID NO: 5 - DNA, 31 bp, synthetic construct
  source 1..31: mol_type = other DNA
gacaagtgcaagaagcgctacgagaactacc

SEQ ID NO: 6 - DNA, 31 bp, synthetic construct
  source 1..31: mol_type = other DNA
ggtagttctcgtagcgcttcttgcacttgtc

SEQ ID NO: 7 - DNA, 33 bp, synthetic construct
  source 1..33: mol_type = other DNA
ctacgactgcaagttgaacaagcacgctagatc

SEQ ID NO: 8 - DNA, 33 bp, synthetic construct
  source 1..33: mol_type = other DNA
gatctagcgtgcttgttcaacttgcagtcgtag

SEQ ID NO: 9 - DNA, 27 bp, synthetic construct
  source 1..27: mol_type = other DNA
atctgtgactactgcagatactaagcg

SEQ ID NO: 10 - DNA, 27 bp, synthetic construct
  source 1..27: mol_type = other DNA
tctgcagtagtcacagatgcactgcag

SEQ ID NO: 11 - protein, 54 aa, synthetic construct
QDKCDKVYENYPVSKCQLANQCNYDCKLDKHARSGECFYDEKRNLQCICDYCEY

SEQ ID NO: 12 - DNA, 165 bp, synthetic construct
  source 1..165: mol_type = other DNA
caggacaagtgcgacaaggtctacgagaactacccagtttccaagtgccagttggctaaccagtgtaactacgactgcaagttggacaagcacgctagatctggtgagtgtttctacgacgagaagagaaacctgcagtgcatctgtgactactgcgagtactaa

SEQ ID NO: 13 - protein, 54 aa, synthetic construct
QDKCKKRYENYPVSKCQLANQCNYDCKLDKHARSGECFYDEKRNLQCICDYCEY

SEQ ID NO: 14 - DNA, 165 bp, synthetic construct
  source 1..165: mol_type = other DNA
caggacaagtgcaagaagcgctacgagaactacccagtttccaagtgccagttggctaaccagtgtaactacgactgcaagttggacaagcacgctagatctggtgagtgtttctacgacgagaagagaaacctgcagtgcatctgtgactactgcgagtactaa

SEQ ID NO: 15 - protein, 54 aa, synthetic construct
QDKCKKVYENYPVSKCQLANQCNYDCKLNKHARSGECFYDEKRNLQCICDYCEY

SEQ ID NO: 16 - DNA, 165 bp, synthetic construct
  source 1..165: mol_type = other DNA
caggacaagtgcaagaaggtctacgagaactacccagtttccaagtgccagttggctaaccagtgtaactacgactgcaagttgaacaagcacgctagatctggtgagtgtttctacgacgagaagagaaacctgcagtgcatctgtgactactgcgagtactaa

SEQ ID NO: 17 - protein, 54 aa, synthetic construct
QDKCKKVYENYPVSKCQLANQCNYDCKLDKHARSGECFYDEKRNLQCICDYCRY

SEQ ID NO: 18 - DNA, 165 bp, synthetic construct
  source 1..165: mol_type = other DNA
caggacaagtgcaagaaggtctacgagaactacccagtttccaagtgccagttggctaaccagtgtaactacgactgcaagttggacaagcacgctagatctggtgagtgtttctacgacgagaagagaaacctgcagtgcatctgtgactactgcagatactaa

SEQ ID NO: 19 - protein, 54 aa, synthetic construct
QDKCDKRYENYPVSKCQLANQCNYDCKLDKHARSGECFYDEKRNLQCICDYCEY

SEQ ID NO: 20 - DNA, 165 bp, synthetic construct
  source 1..165: mol_type = other DNA
caggacaagtgcgacaagcgctacgagaactacccagtttccaagtgccagttggctaaccagtgtaactacgactgcaagttggacaagcacgctagatctggtgagtgtttctacgacgagaagagaaacctgcagtgcatctgtgactactgcgagtactaa

SEQ ID NO: 21 - protein, 54 aa, synthetic construct
QDKCKKVYENYPVSKCQLANQCNYDCKLNKHARSGECFYDEKRNLQCICDYCRY

SEQ ID NO: 22 - DNA, 165 bp, synthetic construct
  source 1..165: mol_type = other DNA
caggacaagtgcaagaaggtctacgagaactacccagtttccaagtgccagttggctaaccagtgtaactacgactgcaagttgaacaagcacgctagatctggtgagtgtttctacgacgagaagagaaacctgcagtgcatctgtgactactgcagatactaa

SEQ ID NO: 23 - protein, 54 aa, synthetic construct
QDKCDKRYENYPVSKCQLANQCNYDCKLNKHARSGECFYDEKRNLQCICDYCRY

SEQ ID NO: 24 - DNA, 147 bp, synthetic construct
  source 1..147: mol_type = other DNA
caggacaagtgcgacaagcgctacgagaactacccagtttccaagtgccagttggctaaccagtgtaactacgactgcaagttgaacaagcacgctagatctggtgagtgtttctacgacgagaagagaaacctgcagtgcatctgt